(19) United States
(12) Patent Application Publication
(10) Pub. No.: US 2003/0198632 A1
(43) Pub. Date: Oct. 23, 2003

(54) Thermolysin enzymatic wound debrider

(75) Inventors: Lei Shi, San Antonio, TX (US); David P. Jones, San Antonio, TX (US); Robert Espinoza, San Antonio, TX (US); Braham Shroot, San Antonio, TX (US)

Correspondence Address: MCKEE, VOORHEES & SEASE, P.L.C., 801 GRAND AVENUE, SUITE 3200, DES MOINES, IA, 50309-2721, US

(73) Assignee: Healthpoint, Ltd., Fort Worth, TX

(21) Appl. No.: 10/304,929

(22) Filed: Nov. 26, 2002

Related U.S. Application Data
(62) Division of application No. 10/124,958, filed on Apr. 18, 2002.

(51) Int. Cl. A61K 38/48

(52) U.S. Cl. 424/94.63

(57) ABSTRACT

An enzymatic wound debrider which employs in combination a topical dosage form pharmaceutical carrier and a debridement agent which is thermolysin preferably in combination with an ionic co-factor of either zinc ions, or calcium ions, or both.

### FIELD OF THE INVENTION

This invention relates to a composition for the enzymatic debridement of necrotic tissue, and for liquefaction of pus in acute and chronic wounds.

### BACKGROUND OF THE INVENTION

Enzymatic wound debridement has been known in the past. However, enzymatic debriders have had substantial stability and efficacy problems. In particular, prior art wound debridement compositions normally require refrigeration for stable storage, and have shelf life stability problems because the current used enzymes show degradation by protein denaturation due to poor conformational stability. Water has been thought necessary because it allows dissolving of the enzyme, which in turn was thought necessary in order to have effective wound debridement. Enzymes with poor thermal stability will more likely unfold or be oxidized in water, thus lose activity. Further, the current applied cysteine-proteases and serine-proteases are very broad in enzymatic specificity. The non-specific enzymatic action will result in elevation of the dose charge causing more irritation. These enzymes show very poor efficacy to digest collagen type wound proteins.

Debridement agents are those agents which rapidly digest necrotic tissue without injury to living cells, thereby speeding the healing processes. The search for such debridement agents has included the employment of a wide variety of plant and animal materials, even things such as maggots or blowfly larvae, but more commonly, the enzyme papain derived from the papaya tree, and the enzyme trypsin derived from animal pancreas. The mechanism in almost all of these cases has been identified with enzymatic activity.

Healing of wounds is delayed by the presence of pus, tissue debris, bacteria, and exudates. The primary purpose of the debriding enzyme is to clean a wound of all of the various necrotic tissue elements and to thin out thick exudative secretions. When properly applied to selected patients, certain proteolytic enzymes cleanse infected surfaces of their inflammatory exudate without harm to living tissues; facilitate the drainage of areas of located purvulent, sanguineous and fibrinous accumulations; promote the liberation of hidden bacteria, thereby exposing them to antimicrobial agents and native immune forces, and increase the rate of repair of previously infected wounds. This enzymatic action can also be of benefit for the treatment of inflammatory skin diseases such as psoriasis and eczema.

Perhaps the most commonly used debridement agents of those earlier referenced, are those using non specific proteases such as papain. While papain has proved somewhat effective in the past, it does have its own storage problems, inherently associated with the properties of papain. For example, papain is quite heat sensitive and the presence of certain metal ions are known to inhibit its activity. Papain also has a characteristic odor. Therefore, there has been a continuing effort to find better wound debridement enzymes. Some of the criteria for a highly preferred wound debridement enzyme are the following: it should be capable of rapid digestion of fibrin, denatured collagen, elastin and exudate; it should spare normal appearing human skin tissues; it should be non-toxic and non-irritating to wounds; it should be easily prepared, stable and readily applicable in most situations; it should have minimum sensitivity to temperature; it should have low odor; it should have good shelf life stability and ideally it should not be significantly inhibited by the presence of certain metal ions, particularly, those which participate in wound healing, like calcium and zinc. This invention has as its primary objective the furtherance of the goal of more closely meeting the criteria of an ideal wound debridement agent as above described. In particular, there is a continuing need for a development of enzymatic wound debriders, which show good efficacy for debriding necrotic tissue, which are thermally stable, which have low odor, which have good shelf life stability and which are not desensitized by wound healing co-factors such as zinc and calcium ions. This invention fulfills that need.

The method and manner of accomplishing the above primary objective, as well as others, will become apparent from the detailed description of the invention which follows.

### BRIEF DESCRIPTION OF THE DRAWINGS

FIGS. 1-3 show a elastolysis, fibrinolysis, and collagenolysis for a 1% thermolysin cream when tested for in vitro efficacy, comparing with a papain/urea debrider.

FIG. 4 shows the stability testing of the thermolysin cream of FIGS. 1-3, at room temperature and accelerated temperature (40° C.).

FIGS. 5-8 show comparison efficacy studies of thermolysin in solution on four fibrous proteins of common eschar tissue, in comparison with trypsin and papain under similar circumstances.

### SUMMARY OF THE INVENTION

An enzymatic wound debrider which employs in combination a topical dosage form pharmaceutical carrier and a debridement agent which is thermolysin. The wound debrider may also employ in combination with the thermolysin, an ionic co-factor of a metal ion. The metal ions may either be zinc ions, or calcium ions, or both.

### DETAILED DESCRIPTION OF THE PREFERRED EMBODIMENT

Thermolysin is a known enzyme. For details see Chapter 351 of Handbook of Proteolytic Enzymes, pages 1037-1046. There it is reported that the thermolysin activity is inhibited by metal-chelating agents. e.g.(page 1040) it is reversibly inhibited by zinc-binding agents such as EDTA, a phosphoramidate group, a sulthydryl group, or a hydroxamate group. However, calcium chloride in the range of 1-10 mM is usually added in buffers to minimize autolysis.

Thermolysin is an extracellular, 34.6 kD metalloendopeptidase secreted by the gram-positive thermophilic bacterium Bacillus thermoproteolyticus. It is a thermostable protease with very potent proteolytic activities towards the wound proteins, i.e. collagen and fibrin, in necrotic tissues. Comparing thermolysin with other commonly used wound debridement proteases, papain or trypsin, it demonstrates higher collagenolysis and fibrinolysis and shows excellent efficacy in wound debridement. Unlike those other enzymes, thermolysin is not inhibited by metal ions but rather works with metal ions such as calcium ions and zinc ions to stabilize the structure for its function. Metal ions usually interact with the functional groups at the active sites of cysteine or serine proteases like papain and trypsin. However, thermolysin favors calcium and zinc ions for optimizing conformational stabilization. This is important since those ions also happen to function as co-factors to enhance healing. Other metal ions which may also be present with thermolysin include cobalt, copper, manganese, iron, nickel and cadmium. Thermolysin shows high thermostability capable of working up to 80° C. Improved thermostability, when achieved, allows for preparations with thermolysin to be stored at room temperature instead of at cool or refrigeration temperatures as is the case for some other enzymatic preparations. A longer shelf life of the thermolysin preparation can also be realized. In addition, the thermolysin will withstand higher processing temperatures than will other enzymes when manufacturing the preparation.

A variety of pharmaceutically acceptable preparations familiar to those skilled in the art could be utilized as a vehicle for the thermolysin. Preparations such as creams, lotions, ointments, gels, solutions, suspensions, sprays, aerosol sprays, aerosol foams and mousses, or thermo reversible preparations could be used.

An effective amount of the enzyme is to be used in the practice of this invention. Such amount will be that amount which effectively debrides necrotic tissue and which liquefies pus in acute and chronic wounds. Such an amount will also be that amount which effects removal in a reasonable time (for example, over a 7 day period), of substantially all of such materials. The precise amount used for any particular use will depend on several factors, including the inherent activity of the enzyme, the number of applications intended for the wound, etc. In weight/volume terms, the enzyme preparations are seldom pure, and it is expected that the enzyme source will be used in amounts of from 0.001% to 15% of the weight of the total formulation. Precise amounts will vary with purity of the enzyme.

The type of topical dosage form ointment or base pharmaceutical carrier utilized will depend, of course, to some extent upon the nature of the area to be treated. In general, almost any pharmaceutical topical ointment or base which does not inactivate or interfere with the enzymatic action, may be employed.

Other pharmaceutical dose forms such as packets of the enzyme product can be prepared which allow for the extemporaneous preparation of lotions, etc. if for some reason the ointment or jelly form is unacceptable. Suitable topical water-based pharmaceutical carriers would be known to one of ordinary skill in the art. For an anhydrous topical pharmaceutical carrier, see co-pending and commonly assigned application Hobson et al., Ser. No. 09/749,217 filed Dec. 27, 2000, the disclosure of which is incorporated herein by reference.

As those skilled in the art know, the compositions of the present invention may contain other components referred to as minors such as enzymatic stabilizers, wound healing agents such as copper chlorophyllin complex and its salts, vitamins such as vitamin A and vitamin D, antioxidants such as lipoic acid, structure-forming ingredients, anti-microbial agents, antibiotic agents, and/or anesthetic agents, all generally from the GRAS safe list. Generally, amounts of these will vary from 0.01% to 25%.

The following examples are offered to further illustrate but not limit the invention.

### EXAMPLE 1

Enzymatic activities of thermolysin on substrates of fibrin, elastin, gelatin and collagen were tested. Results appear below but generally thermolysin shows higher potencies to digest these proteins than the comparison enzymes of papain and trypsin. In particular, a thermolysin cream containing 1% thermolysin was made. The pharmaceutical formulation was the following:

| Raw Material | Percentage, w/w |
|---|---|
| Emulsifying Wax | 11.0 |
| Isopropyl Palmitate | 4.0 |
| Propylparaben | 0.08 |
| Glycerin | 5.0 |
| Methylparaben | 0.2 |
| Potassium Phosphate Monobasic | 1.0 |
| Thermolysin | 1.0 |
| Calcium Acetate | 1.0 |
| Zinc Acetate | 2.0 |
| Water | 74.72 |

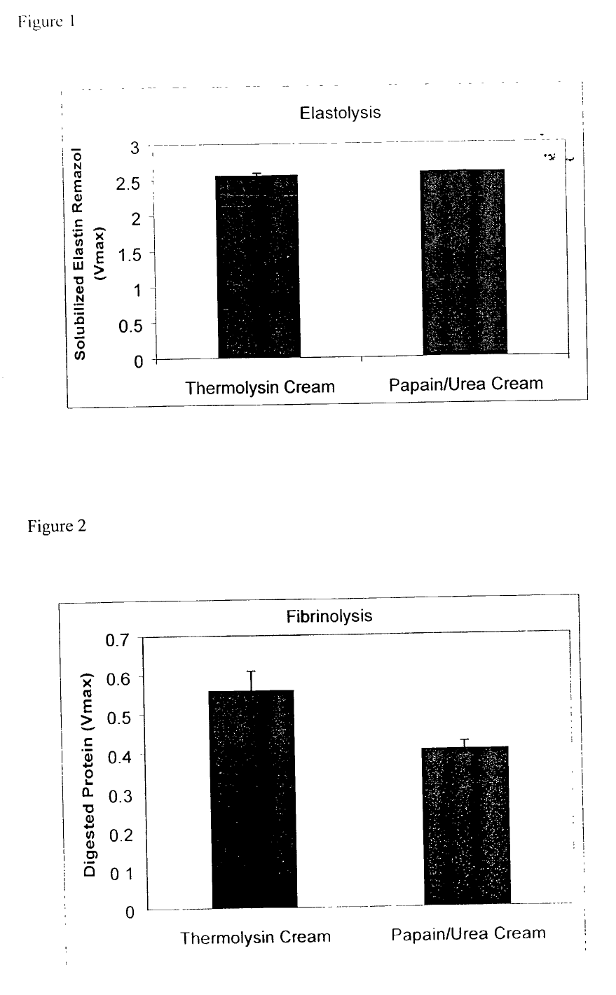

The formulated 1% thermolysin cream was also tested for its in vitro efficacy. The results are shown in the FIGS. 1-3.

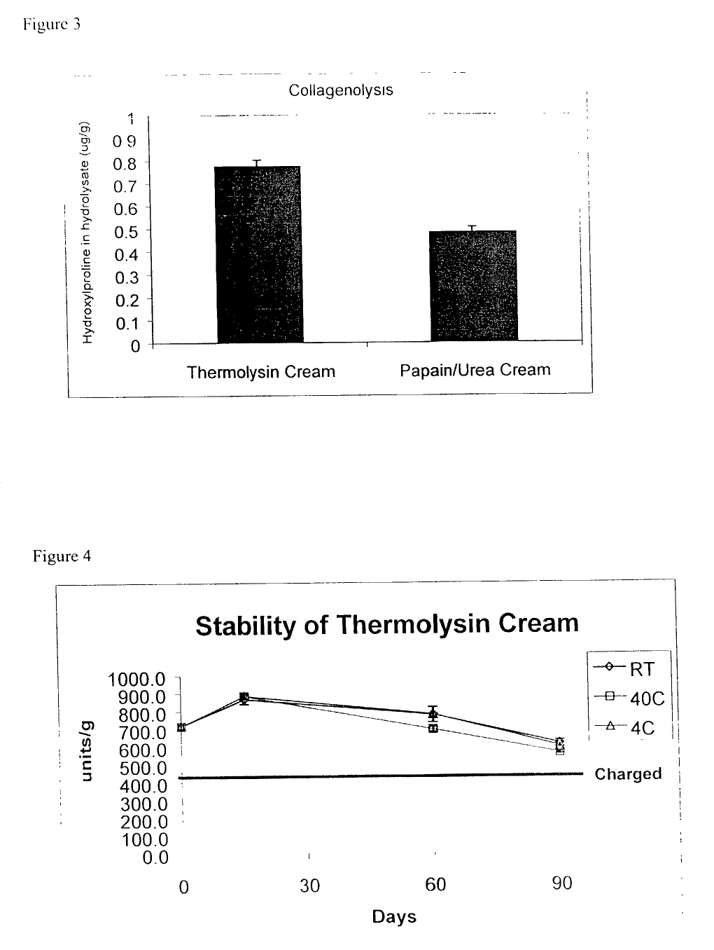

The formulated thermolysin cream showed very good stability even when it was stored at 40° C. The cream was stored for ninety days with FIG. 4 showing the activity of the thermolysin cream during the first ninety days of storage, both at room temperature and at 40° C.

These results demonstrate the superiority of thermolysin using zinc ion and calcium ion as co-factors.

When calcium and zinc ion salts are used as co-factors, their percentage level in the ultimate formulation should vary from 0.001 to 15.0, and preferably from 0.01 to 3.0.

EXAMPLE 2

| Raw Material | Percentage, w/w |
|---|---|
| Emulsifying Wax | 15.0 |
| Isopropyl Palmitate | 6.0 |
| Propylparaben | 0.05 |
| Glycerin | 10.0 |
| Methylparaben | 0.25 |
| Thermolysin | 0.2 |
| Calcium Chloride | 1.0 |
| Zinc Chloride | 0.2 |
| Water | 67.3 |

EXAMPLE 3

| Raw Material | Percentage, w/w |
|---|---|
| Poloxamer 407 | 10.0 |
| Poloxamer 338 | 18.0 |
| Poloxamer 124 | 69.8 |
| Thermolysin | 1.0 |
| Calcium Acetate | 1.0 |
| Zinc Acetate | 0.2 |

Efficacy studies of thermolysin on the four fibrous proteins of common eschar tissue were conducted. In particular, thermolysin was compared with papain. The dry thermolysin was labeled 8,700 U/mg in 11.8% sodium acetate and 22.7% calcium acetate. With a standard papain USP method, it was determined to have 55,083 USP U/mg on a casein substrate.

The papain was high purity papain with 51,780 USP U/mg.

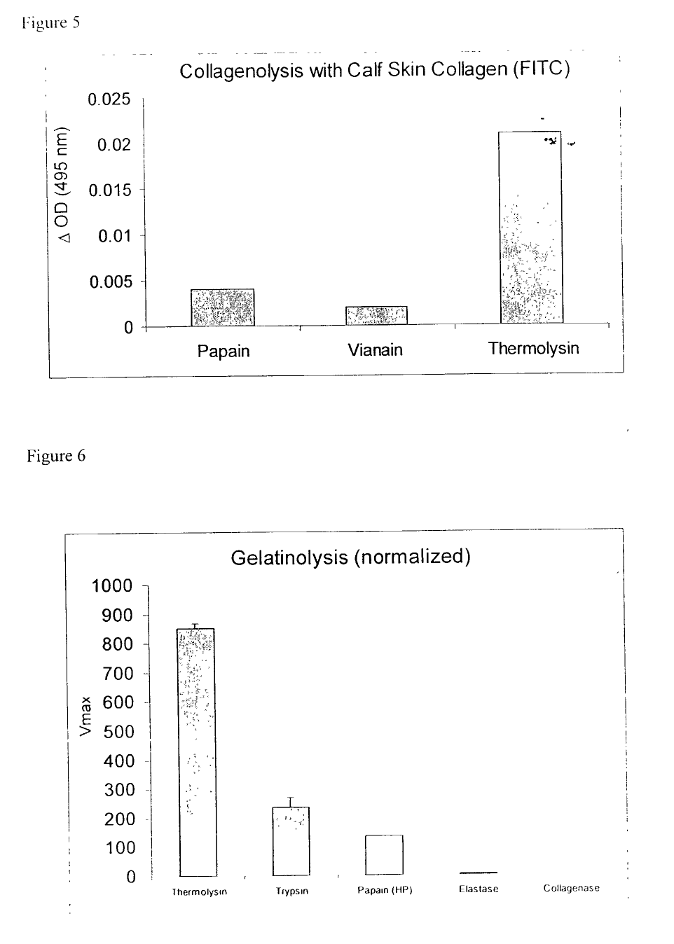

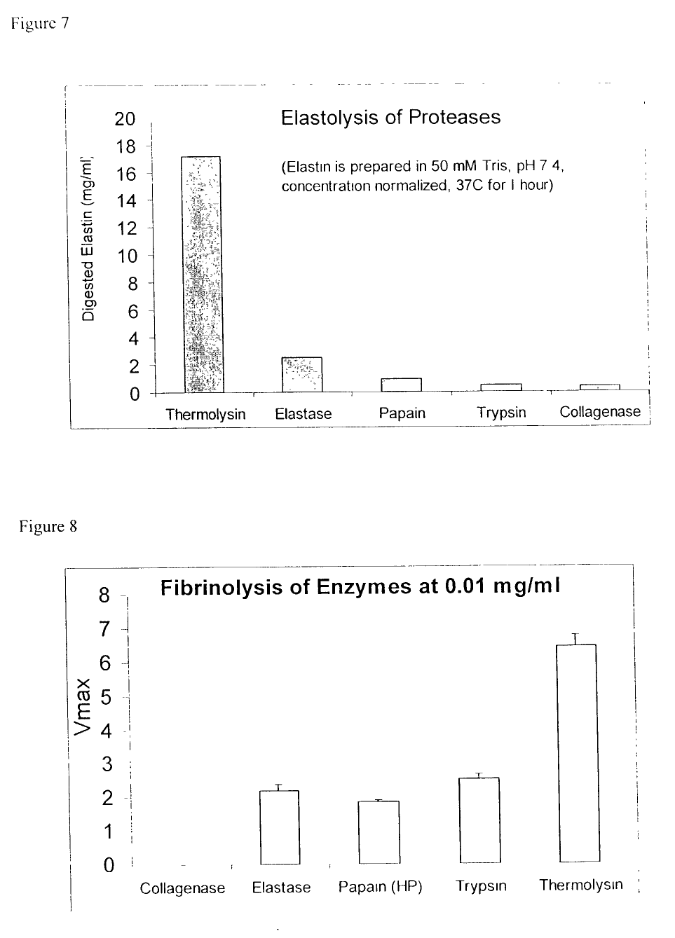

The substrates upon which the test were conducted were collagen (EPC Collagen-FITC); gelatin (sigma porcine skin gelatin); elastin (EPC Elastin-Remazol); and fibrinogen for making fibrin (Calbiochem). A buffer solution of 50 mM tris, 100 mM NaCl, 10 mM CaCl₂, pH=7.4. FIGS. 5, 6, 7 and 8 show comparisons of thermolysin with other proteases and demonstrate clearly the superiority of thermolysin.

### EXAMPLE 5

Thermolysin creams containing 1% of example 1 and 0.2% of example 2 thermolysin were applied to necrotic tissues on pigs for in vivo debridement efficacy study. Approximately 0.5 g of each thermolysin cream, together with a papain/urea debrider, was used to each of the generated wound (about 2 cm in diameter). After 24 hours, significant wound debridement was observed on the wounds treated with the thermolysin creams. After 5 days, those with thermolysin cream showed clean surface without any necrotic tissue and complete healing. Papain/urea debrider also showed significant debridement after 48 hours. However, the wounds were not as clean as those treated with thermolysin creams, and did not show complete healing after five days.

From the above examples which are illustrative of the invention, it can be seen that the invention accomplishes its objectives and fulfills the need earlier described. It goes without saying that modifications and additions may be made to the described formulations without departing from the invention.

What is claimed is:

1. An enzymatic wound debrider composition, comprising:
a topical dosage form pharmaceutical carrier; and
a small but debridement effective amount of thermolysin.

2. The enzymatic wound debrider of claim 1 wherein the amount of enzyme is from about 0.001% to about 15% by weight of the composition.

3. The enzymatic wound debrider of claim 1 which has a metal ion co-factor.

4. The enzymatic wound debrider of claim 3 wherein the metal ion co-factor is selected from the group consisting of zinc ions and calcium ions.

5. The enzymatic wound debrider composition of claim 3 wherein the metal ion co-factor is a salt in a concentration of 0.001% by weight to 15.0% by weight.

6. The enzymatic wound debrider composition of claim 5 wherein the metal ion co-factor salt has a concentration of 0.01% by weight to 3.0% by weight.

7. The enzymatic wound debrider composition of claim 1 wherein the pharmaceutical carrier is an anhydrous carrier.

8. The enzymatic wound debrider composition of claim 7 wherein the pharmaceutical carrier is a poloxamer carrier.

9. The enzymatic wound debrider of claim 1 which has an anti-microbial agent.

10. The enzymatic wound debrider of claim 1 which has a wound healing agent.

11. A method of enzymatic wound debridement, comprising:
applying topically to a wound in need of debridement; a topical dosage pharmaceutical carrier containing a small but debridement effective amount of thermolysin.

12. The method of enzymatic wound debridement of claim 11 wherein the carrier also contains a metal ion co-factor.

13. The method of enzymatic wound debridement of claim 12 wherein the metal ion co-factor is selected from the group consisting of zinc ions and calcium ions.

14. A method of enzymatic treatment of inflammatory skin diseases, comprising:
applying topically to the affected area, a topical dosage pharmaceutical carrier containing a small but effective amount of thermolysin.
